(19) United States
(12) Patent Application Publication
(10) Pub. No.: US 2009/0301936 A1
(43) Pub. Date: Dec. 10, 2009

(54) COMPOSITION AND USE THEREOF

(76) Inventors: Desmond Smith, Swindon - Wiltshire (GB); Colin Smith, Staines - Surrey (GB); Duncan Watson, Staines -Surrey (GB)

Correspondence Address: MCANDREWS HELD & MALLOY, LTD, 500 WEST MADISON STREET, SUITE 3400, CHICAGO, IL, 60661, US

(21) Appl. No.: 12/465,397

(22) Filed: May 13, 2009

Related U.S. Application Data
(60) Provisional application No. 61/053,353, filed on May 15, 2008.

(30) Foreign Application Priority Data
May 15, 2008 (GB) 0808877.5
Jun. 6, 2008 (GB) 0810409.3

(51) Int. Cl. C10G 29/20; C08G 69/26; C08L 77/06; G01N 33/00; C09K 3/00

(52) U.S. Cl. 208/289; 524/612; 524/606; 525/432; 436/128; 252/182.29

(57) ABSTRACT

The present technology relates to a method of treating hydrocarbon compositions such as crude oils to reduce or prevent naphthenate soap problems in upstream and downstream processes. It also relates to compositions for this use, for example.

### RELATED APPLICATIONS

This non-provisional application claims priority to (1) U.S. Provisional Application No. 61/053,353, filed on May 15, 2008; (2) Great Britain Application No. 0808877.5, filed May 15, 2008; and (3) Great Britain application No. 0810409.3, filed Jun. 6, 2008. The present application hereby incorporates U.S. Application No. 61/053,353 and Great Britain Application Nos. 0808877.5 and 0810409.3 in their entireties.

### BACKGROUND OF THE INVENTION

The present technology relates to a method of treating hydrocarbon compositions such as crude oils to reduce or prevent naphthenate soap problems in upstream and downstream processes. It also relates to compositions e.g. for this use.

The energy sector has been experiencing discoveries of increasing numbers of oil reservoirs being found and developed since the late 1990s that eventually yield a medium to heavy oil type as classified by API charts and recognizable by one skilled in the art (i.e. oils with API gravities of between 15 to 25°). At one time these heavier fields would have been developed on second priority basis. However, sustained high oil prices, and other factors, mean that these new oilfields have become viable.

A typical characteristic for these oil types is that the crude oil may show an appreciable amount of naphthenic acid content. This may be deduced and estimated from its acid value. The acid value of a crude oil is termed its Total Acid Number (TAN) value (mg KOH/g oil). However, the TAN values do not give a prediction of the risk of forming naphthenate soap solids or other soap types during reservoir fluid production in oilfields, nor in refinery heat exchangers.

Naphthenic acid problems including naphthenate soap deposit problems have been experienced for at least fifty years in the oil industry.

A naphthenic acid in the oilfield is considered as a carboxylic acid with a mono or dibasic carboxy group(s) attached to a cycloaliphatic structure. In this patent application we define a naphthenic acid as any organic acid in a crude oil. This complies with the Energy sector which considers that all organic acids in crude oil are called naphthenic acids. Naphthenic acids in crude oils are mixtures of thousands of component molecules from low to high molecular weight organic acids. These acids can be very water soluble to oil soluble depending on their molecular weight, on the process temperatures their parent fluids are subjected to, on the salinity and pH of their parent waters, and finally on their parent fluid processing pressures [1, 2, 5 & 6]. This property is revealed in the acids partitioning character to gas, oil and water under the operating conditions. Herein naphthenic acid includes linear acids, carboxylic acids, cyclic aliphatic acids and aromatic acids.

Naphthenic acid nomenclature has been known to be difficult to rationalize in the Energy sector going back almost 100 years when the acid was found in crude oils [12]. The “enic” in its name implied unsaturation, in the old sense of meaning a double bond. But the name is really accepted as one meaning a depletion of hydrogens on the carbon atom thus leading to cycloaliphatic structures. Also we consider napthenic acid to cover aromatic acids such as a set of carboxylic acids found in heavy Californian crude oils known to yield naphthenic and naphtheno-aromatic acids, for example [13, 14, 15].

Naphthenic acids in crude oils and refinery product streams vary in molecular weights from approximately 100 to >1300 molecular weight (MW), as published in mass spectrometric (MS) and Fourier Transform Ion Cyclotron Resonance (FTICR) analyses [6,7]. In a problematic crude oil there is likely to be a certain percentage of host reservoir water included with the oil. These waters contain brine which has significant metal content, but most relevant here are the alkali metals, the alkaline earth metals, and iron and aluminum metal ions in solution. The naphthenic acids have the potential to react with these metals and minerals, which can neutralize the acids to form insoluble salts and hence the naphthenate soaps. A soap in this patent application is defined as a substituted carboxylic acid neutralized or covalent bonded by a cation with the cation acting as an alkali. A naphthenate soap herein is a soap formed from a napthenic acid.

The calcium naphthenic acid soap, known to the oil industry as calcium naphthenate, is a major component of the mixed soaps, but analyses of these deposits from various oilfield worldwide reveal that they are often a mixture of one or more soap (including calcium, magnesium, sodium, potassium, iron, and aluminum), with occluded silica/sand, mineral scales, iron hydroxides/oxides and sulphides, formation clay, mud residues e.g. bentonites, flocculated asphaltenes, paraffin waxes, resins and treating chemicals [5, 6, 8]. These soaps and other occluded particles have been known under real conditions for a long time [16,]. These adhere to oil water interfaces and can become finely dispersed within the water itself. The oil industry to date has solved the emulsion problems caused by naphthenate soaps using acidic demulsifiers, called “pad busters” or interface draw-offs to separate slop treatments [2 to 6 & 8]. The reverse emulsions in oils and in dirty oilfield water are being treated with water clarifiers, most of these being the cationic water clarifiers [5, 8].

Naphthenate soaps can be found across the entire oil process system from wellheads, subsea flowlines, well manifolds, separators, desalting equipment, heat exchanger tubes, crude oil and produced water cooling equipment to oil filters and even within oil storage tanks. These deposits can harden to cement-like structures requiring frequent system shutdowns. The procedure to clean these deposits off the vessels involves acid dissolving techniques and physical sonic, mechanical breakup and vibration methods. Shutting down a system or crude oil train for clean-up periods, costs millions of dollars in maintenance costs and lost production [5]. Excess naphthenate soap micelles as residual soaps, dispersed in exported crude oils to refineries can sludge out in terminal tanks and cause fouling in lines, desalters and heat exchanger units. These naphthenate soaps can cause crude oil reception tank problems, and may affect the drainage of water in the tank farms. Residual emulsified water may affect the desalters, earring alkali and alkaline earth metals to the heat exchangers and furnaces and, with the high heat applied in these ancillary units, metals react with the free naphthenic acids to form soaps which can lead to heat exchanger fouling. Naphthenate soaps can also fragment in the high temperatures used in the crude distillation units to form carbon dioxide, low molecular weight naphthenic acids, leading to both corrosion and scale and catalyst poisoning.

Most published approaches to naphthenic acids in the oilfield involve GC-MS work, Infra Red, and FT-ICR. ExxonMobil has published an infra red method for analysis and estimation for blending crude oil slates mainly for corrosion, and also published two removal processes involving adding inert materials to remove the acids then decanting the weak complexes of higher densities than crude oils to the bottom of the vessel. The other method for removal of the naphthenic acids is in crude in a refinery whereby the acids are complexed on a modified resin. These processes have not reached the upstream sector.

Although some crude oils may not give naphthenate soap fouling in the oilfield e.g. crude oils from the Chad African fields, these crude oils do cause severe fouling in the refinery. These oils are known as high calcium in crude oils. Nalco has developed a chemical method for removal of the Calcium from such crude oils.

Not all the naphthenic acids in a crude oil react to form complexed naphthenate soaps in the oilfield in the dynamic conditions involved and the retention time available. Even a crude oil that deposits heavy naphthenate soaps will still retain a TAN value, due to residual naphthenic acids. Thus, there is a also need for prevention of naphthenate soap formation in refineries.

There are no published methods to inhibit the formation of naphthenate soaps via a chemical injection to control naphthenic acids to naphthenate soap reaction/formation in the oilfield, or naphthenate soap formation in refineries due to the residual napthenic acids in the exported crude oil.

### BRIEF SUMMARY OF THE INVENTION

The present technology provides a composition comprising at least one component and at least one dispersant where the component has a Total Base Number of 50 mg KOH/g or above, and a substituted primary amine. Certain embodiments of the present technology provide at least one composition wherein the substituted primary amine is of the general formula:

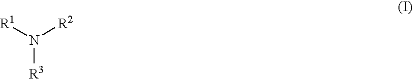

wherein R¹, R² are Hydrogen (H), and R³ is a linear or branched alkyl chain. Certain embodiments of the present technology present methods for using and treating the composition described herein.

### BRIEF DESCRIPTION OF SEVERAL VIEWS OF THE DRAWINGS

[Not Applicable]

### DETAILED DESCRIPTION OF THE INVENTION

According to the present technology in a first aspect there is provided a composition comprising a component having a Total Base Number (TBN) of 50 mg KOH/g or above and including a substituted primary amine; and a dispersant. The composition may further comprise a solvent. The component having a Total Base Number (TBN) of 50 mg KOH/g or above may have Total Base Number (TBN) of, for example, between 50 and 800 mg KOH/g, for example between 60 and 700 mg KOH/g, for example between 100 and 350 mg KOH/g, e.g. TBN of between 200 and 350 mg KOH/g, e.g. between 260 and 350 mg KOH/g. The ratio of primary amine:dispersant may be between (% by weight) 90:10 and 60:40, e.g. 80:20 to 70:30. The ratio of solvent:(dispersant plus primary amine) may be between 90:10 and 60:40.

The applicants have found that the compositions according to the present technology may surprisingly inhibit and/or reduce naphthenate soap and/or other solid formation by (e.g. caused by or due to) reaction of naphthenic acids (e.g. in upstream and downstream crude oil processes). The applicants have found that the compositions according to the present technology may surprisingly inhibit and/or reduce naphthenate soap and/or other solid formation e.g. by inhibiting the soap forming action of the naphthenic acids. Further, the applicants have found that the compositions according to the present technology which include a dispersant may help disperse (e.g. reduce the amount of) and/or break down pre-existing naphthenate soaps (i.e. break down naphthenate soaps formed, or present in a hydrocarbon composition/stream, prior to addition of the composition). Removal or break down of pre-existing soaps may further improve the inhibition and/or reduction of (further) naphthenate soap formation. The composition(s) of the present technology may be used to treat hydrocarbon compositions, e.g. including crude oil, partially purified crude oil or oil(s) or substance(s) obtained from crude oil following subsequent crude oil distillation, for example petroleum, kerosene, or paraffin. The composition(s) may be used to treat samples obtained from crude oil directly, or from sludges, oil deposits, oil emulsions, or tars which have been prepared for chemical injections. The composition(s) is preferably applied via notched quill injector or atomiser injection.

The component including a substituted primary amine has a Total Base Number (TBN) of 50 mg KOH/g or above. It may, for example, have Total Base Number (TBN) of between 50 and 800 mg KOH/g, for example between 60 and 700 mg KOH/g, for example between 100 and 350 mg KOH/g, e.g. TBN of between 200 and 350 mg KOH/g, e.g. between 260 and 350 mg KOH/g. Total Base Number (“TBN”) terminology is well known in the art; TBN is measured in milligrams of potassium hydroxide per gram (mg KOH/g).

The composition of the present technology [including the component having a Total Base Number (TBN) of 50 mg KOH/g or above (comprising primary amine and optional booster amine); dispersant and optional solvent] may have, overall, a Total Base Number (TBN) of 50 mg KOH/g or above. It may, for example, have Total Base Number (TBN) of between 50 and 800 mg KOH/g, for example between 60 and 700 mg KOH/g, for example between 100 and 350 mg KOH/g, e.g. TBN of between 200 and 350 mg KOH/g, e.g. between 260 and 350 mg KOH/g. The composition may, for example, include a component having a Total Base Number (TBN) of, for example, between 100 and 350 mg KOH/g, e.g. TBN of between 200 and 350 mg KOH/g, e.g. between 260 and 350 mg KOH/g (the component comprising primary amine and optional booster amine); dispersant; and optional solvent; and may have, overall, a Total Base Number (TBN) of 50 mg KOH/g or above.

The substituted primary amine may be an amine of general formula:

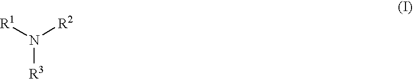

in which R¹, R² are Hydrogen (H), and R³ is a linear or branched alkyl chain having, for example, between 1 and 100 Carbon atoms and which may optionally be substituted and which may optionally include heteroatoms (e.g. P, N, O, S). Herein, groups which are optionally substituted may be substituted by up to five groups independently selected from, for example, an —OH group, an amine group, a (C₁-C₁₀)alkyl group, a (C₃-C₁₀)cycloalkyl group, a (C₂-C₁₀)alkenyl group, a (C₃-C₁₀)aryl group, and a (C₃-C₁₀)heterocylic group.

The substituted primary amine may be an amine of general formula:

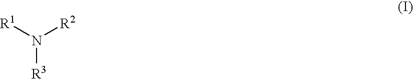

in which R¹, R² are Hydrogen (H), and R³ is selected from a polyalkylene-polyamine and a polyalkylene-polyamine-hydrocarbyl succinimide residue. R³ may be a polyalkylene-polyamine-hydrocarbyl succinimide residue of formula R⁴—CH₂—CH₂—(NH—CH₂—CH₂)n wherein R⁴ is a polyisobutenyl succinimide (PIBS) residue, and n is between 1 and 15, for example between 1 and 10, for example n=1 or n=2.

The substituted primary amine may be a modified tris succinimide of general formula:

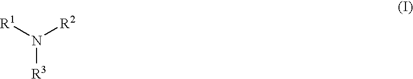

wherein R¹, R² are each of formula R⁴—CH₂—CH₂—(NH—CH₂—CH₂)ₙ wherein R⁴ is a polyisobutenyl succinimide (PIBS) residue and n is between 1 and 10; and R³ is of formula R^{4′}—(NH—CH2-CH2-NH—CH2-CH2-)n, R^{4′} is H (so R³ is a modified polyisobutenyl succinimide (PIBS) residue having a free terminal primary amine (NH₂) group, and n is between 1 and 10.

The substituted primary amine may be that supplied under the Product name—FOA-8, code D0350 by Innospec Limited, Innospec Limited Manufacturing Park, Oil Sites Road, Ellesmere Port, Cheshire, CH 65 4EY, United Kingdom. The substituted primary amine may be prepared from a substituted polyamine called a polymeric amine and classed as polyalkylene polyamine. The substituents on the substituted primary amine can take the form of one end being a mono polyisobuylene succinimide and the other end being a free amine.

The component having a Total Base Number (TBN) of 50 mg KOH/g or above and including a substituted primary amine may, for example, comprise a substituted primary amine (or mixture of substituted primary amines) having a Total Base Number (TBN) of 50 mg KOH/g or above. The component having a Total Base Number (TBN) of 50 mg KOH/g or above and including a substituted primary amine may, for example, comprise a substituted primary amine (or mixture of substituted primary amines) having a Total Base Number (TBN) of, for example, between 50 and 800 mg KOH/g, for example between 60 and 700 mg KOH/g, for example between 100 and 350 mg KOH/g, e.g. TBN of between 200 and 350 mg KOH/g, e.g. between 260 and 350 mg KOH/g. The component having a Total Base Number (TBN) of 50 mg KOH/g or above and including a substituted primary amine may, in another example, comprise a substituted amine having a Total Base Number (TBN) of less than 50 mg KOH/g, and may include a further booster amine or amines which increase the TBN value to 50 mg KOH/g or above. The component having a Total Base Number (TBN) of 50 mg KOH/g or above and including a substituted primary amine may, in another example, comprise a substituted amine having a Total Base Number (TBN) of less than 200 mg KOH/g, (e.g. less than 150 mg KOH/g e.g. less than 100 mg KOH/g) and may include a further booster amine or amines which increase the TBN value to e.g. 100, 150 or 200 mg KOH/g or above. If a booster amine (or amines) is present in the component the ratio by weight of primary substituted amine:(total booster amines) may be between 5:95 and 95:5, e.g. between 30:70 and 70:30. The ratio of primary amine:dispersant may be between (% by weight) 90:10 and 60:40, e.g. 80:20 to 70:30.

The booster amine may be of the formula

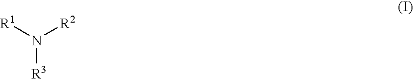

wherein R¹, R² are Hydrogen (H), R³ is of formula R⁴—(NH—CH₂—CH₂)n, R⁴ is H and n is in the range 1-10. For example the booster amine may be triethylene triamine (TETA) if R⁴ is —CH₂—CH₂—NH₂ and n is 3 and DETA if R⁴ is H and n is 2. A preferred booster amine is of formula (I) wherein R¹, R² and R⁴ are Hydrogen, R³ is of formula R⁴—(NH—CH2-CH2)n, and n=4 [Tetra ethylene pentamine (TEPA)]. The booster amine may include methylamine and/or polyamine. The booster amines may be obtained from Aldrich—e.g. Diethylene triamine (DETA) is Ref number: D9, 385-6; Triethylene tetramine (TETA) is Ref # 13, 209-8; and Tetraehylene pentamine (TEPA) is Ref # T1150-9.

The substituted amine and/or the booster amine may be a (C₁-C₁₀)-alkoxylated polyamine, for example a (C₁-C₁₀)-alkoxylated(C₁-C₁₀)alkylamine, e.g. an ethoxylated or propoxylated alkyl amines).

The dispersant may be any dispersant known in the art. Preferably, the dispersant is a bis-succinimide, for example a bis-succinimide of general formula

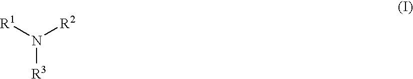

wherein R¹ is Hydrogen (H), R² and R³ are each of formula R⁴—CH2-CH2-(NH—CH2-CH2-)n wherein R⁴ are polyisobutenyl succinimide (PIBS) residues and n is from 1 to 10. The dispersant may also function as a booster amine, in which case it may raise the TBN of the overall composition.

The solvent may be an industrial aromatic solvent, e.g. Solvesso® aromatic grade, toluene or xylene, or mixtures thereof. The solvents may include straight chain or branched C1-C10 alkanes. The term C₁-C₁₀ herein includes the groups methyl, ethyl, propyl, butyl, pentyl, hexyl, heptyl, octyl, nonyl, and decyl. The alkane may be pentane, hexane, and or heptane. Preferably, xylene may be used as a co-solvent. A preferred solvent is Solvesso®/xylene in the % ratio of from 0:95 to 100:5. (Solvesso® is a trade mark of ExxonMobil Corporation). The Solvent: inhibitor may be, for example, in % range solvent: inhibitor 90-60 solvent to 10 to 40 Inhibitor.

If a booster amine is present the Solvent: % range is between 50:50 and 90:10 Suitably, to meet efficiency requirements a mixture range will be in % range solvent: (primary amine inhibitor plus booster amine) of 90-60 solvent to 10 to 40 (primary amine inhibitor plus booster amine).

The composition may further comprise one or more of a glycol, diol, triol and Dowanols® including solvents, e.g. for winterisation. (Dowanols® is a trademark of Dow Chemicals).

In a further aspect, the present technology provides the use of an inhibitor composition having a Total Base Number (TBN) of 50 mg KOH/g or above and comprising a substituted primary amine to treat a hydrocarbon composition to inhibit and/or reduce naphthenate soap formation by (e.g. due to e.g. by reaction of) naphthenic acids within the hydrocarbon composition. In a further aspect, the present technology provides the use of an inhibitor composition comprising a substituted primary amine to treat a hydrocarbon composition to inhibit and/or reduce naphthenate soap formation by (e.g. due to e.g. by reaction of) naphthenic acids within the hydrocarbon composition and/or to treat a hydrocarbon composition to reduce the amount of, and/or break down, naphthenate soaps in the hydrocarbon composition. In a further aspect, the present technology provides the use of an inhibitor composition having a Total Base Number (TBN) of 50 mg KOH/g or above and comprising a substituted primary amine to treat a hydrocarbon composition to reduce the amount of, and/or break down, naphthenate soap(s) in the hydrocarbon composition. The inhibitor composition may further comprise a dispersant and/or solvent. The composition, dispersant and/or solvent etc. may be as described above. The Total Base Number (TBN) of the inhibitor composition may be, for example, between 50 and 800 mg KOH/g, for example between 60 and 700 mg KOH/g, for example between 100 and 350 mg KOH/g, e.g. TBN of between 200 and 350 mg KOH/g, e.g. between 260 and 350 mg KOH/g.

In a further aspect, the present technology also provides a method of treating a hydrocarbon composition to inhibit and/or reduce naphthenate soap formation by (e.g. due to e.g. by reaction of) naphthenic acids within the hydrocarbon composition comprising a step of treating the hydrocarbon composition with an inhibitor composition having a Total Base Number (TBN) of 50 mg KOH/g or above and comprising a substituted primary amine. In a further aspect, the present technology also provides a method of treating a hydrocarbon composition to reduce the amount of, and/or break down, naphthenate soap(s) in the hydrocarbon composition comprising a step of treating the hydrocarbon composition with an inhibitor composition having a Total Base Number (TBN) 50 mg KOH/g or above and comprising a substituted primary amine. The inhibitor composition may further comprise a dispersant and/or solvent. The composition, dispersant and/or solvent may be as described above.

According to a further aspect of the present technology, there is provided a method for the preparation of a composition, comprising the steps of:

(i) heating a primary amine inhibitor to reduce its viscosity;

(ii) adding an aromatic solvent;

(iv) dissolving the inhibitor in the aromatic solvent;

(v) checking the viscosity of the final blend; and

(vi) optionally adding a dispersant.

The composition (inhibitor composition) may be injected as discussed above via quills or atomiser systems and monitored using all process variables from the control as one versed in control room operations would normally collect the standard information. On the chemical application the naphthenic acids/naphthenate soaps control is monitored by several methods as standard well known techniques e.g., Rate of chemical converted to ppm, Calcium in oil, % free water, % Emulsion, filtration rate of oil and water in mls per minute, mls v sq root of minute plotted graphically, heat exchanger fouling indices, TAN values by ASTM D664, ppm solids filtered off, oil in water (ppm) values etc.

According to the present technology there is provided a method of detecting and/or quantifying formation of naphthenate soap in a hydrocarbon composition comprising applying a stainless steel substrate to the hydrocarbon composition and measuring the amount of napthenate soap deposited on the stainless steel substrate; optionally including a step of applying an inhibitor composition having a Total Base Number (TBN) of 50 mg KOH/g or above and comprising a substituted primary amine to the hydrocarbon composition (e.g. prior to applying the stainless steel substrate to the hydrocarbon composition). The substrate may be, for example, a stainless steel scale coupon, coupons being well known in the art, a stainless steel probe etc. It will be appreciated that detection or quantification may be by measuring the weight gain of the stainless steel substrate which may be used to calculate rate of napthenate soap deposition. The applicants have found that stainless steel substrates have an affinity for naphthenate soaps which are deposited thereon.

Quantitation of the concentration of naphthenate soap forming naphthenic acids after the inhibition stage, can be performed by non-routine special tests. Analysis of the inhibited emulsion forming naphthenate soaps e.g. acids/naphthenate soaps going to the water phase can also be monitored by non-routine laboratory tests.

Embodiments of the present technology will now be described with reference to the following examples and tables 1-4. The following Examples are provided for the purposes of illustration only and are not to be construed as being limiting on the invention.

### EXAMPLES

In a laboratory project using the method, three crude oils were chosen from different fields with known Naphthenate problems.

### Examples

### (1.1) Preparation of the Inhibitor Composition

25 grams of FOA-8 inhibitor (code D0350 from Innospec Limited, Innospec Limited Manufacturing Park, Oil Sites Road, Ellesmere Port, Cheshire, CH 65 4EY, United Kingdom) were warmed up to 45 deg C. for 1 hour under a nitrogen blanket to prevent oxidation. 75 grams of Solvesso 150® were added to the FOA-8. The mixture was stirred with a metal stirrer until it is a homogenous and clear solution with no visible lumps or gels.

### Example 1.1a

### Preparation of the (Inhibitor) Composition

25 grams of FOA-8 inhibitor (code D0350 from Innospec Limited, Innospec Limited Manufacturing Park, Oil Sites Road, Ellesmere Port, Cheshire, CH 65 4EY, United Kingdom) were warmed up to 45 deg C. for 1 hour under a nitrogen blanket to prevent oxidation. 75 grams of Solvesso 150® were added to the FOA-8.10 g of bis succinamide dispersant (such as that sold under the name “KT1154 Polyisobutylene Bis-succinimide Ashless Dispersant” by JinZhou KangTai Lubricant Additives Co., Ltd., of Liaoning, China, 121013) were added to the mixture. The mixture was stirred with a metal stirrer until it is a homogenous and clear solution with no visible lumps or gels.

### Example 1.1b

### Preparation of the (Inhibitor) Composition

The following describes a mixture of mid TBN PIB Succinimide boosted with addition of the booster amine. 50 grams of 160 TBN mono PIB-Succinimide [obtained as a mono PIB Succinimide of 160 TBN sold under the name HiTEC 4007™ by Afton Chemical, London road, Bracknell, RG 12 2UW, Berkshire, HiTEC is a trade name of Afton Chemical] were added to 25 g of Solvesso 100 in a 200 ml beaker and warmed up to 55 deg C. under a Nitrogen blanket, until the mono PIB Succinimide solution was clear, less viscous and no gels were observed. 40-50 grams of TEPA booster amine sourced e.g., ex Aldrich number T1150-9, were slowly added, over 30 minutes, to the 160 TBN material in the beaker, and gently stirred to give a composition of TBN of approximately 245. Thus, the TBN of the “component having TBN of 50 mg KOH/g or above (comprising primary amine and booster amine, in this example)” is about 245 mg KOH/g. This mixture is now diluted for use by previous example of blending in a 25:75 weight ratio with Solvesso 100. The dilution gives a composition comprising a primary amine (mono PIBH Succinamide) having TBN approximately 61. Thus, the TBN of the “composition having TBN of 50 mg KOH/g or above (comprising component (primary amine and booster amine) in this example; and solvent)” is 61 mg KOH/g.

### Example 1.1c

### Preparation of the (Inhibitor) Composition

The following describes a mixture of mid TBN PIB Succinimide boosted with addition of the booster amine and which also includes a dispersant. 50 grams of 160 TBN mono PIB Succinimide (obtained from Afton Chemical under the name HiTEC 4007™, as above) were added to 25 g of Solvesso 100 in a 200 ml beaker and warmed up to 55 deg C. under a Nitrogen blanket, until the mono PIB Succinimide solution was clear, less viscous and no gels were observed. 30 grams of TEPA booster amine sourced e.g., ex Aldrich number T1150-9, mixed with 20 g bis succinimide dispersant (such as that sold under the name “KT1154 Polyisobutylene Bis-succinimide Ashless Dispersant” by JinZhou KangTai Lubricant Additives Co., Ltd., of Liaoning, China, 121013) were slowly added, over 30 minutes, to the 160 TBN material in the beaker, and gently stirred to give a composition of TBN of approximately between 200 to 280 mg KOH/g (depending on purity of reagents), typically 245 mg KOH/g. Thus, the TBN of the “component having TBN of 50 mg KOH/g or above (comprising primary amine and booster amine, in this example)” is (about) 245 mg KOH/g. This mixture is now diluted for use by previous example of blending in a 25:75 weight ratio with Solvesso 100. The dilution gives a composition comprising a primary amine (mono PIBH Succinamide) having TBN approximately 61.

### (1.2) Preparation of the Crude Oil Samples

A 100 ml sample of each respective crude oil in a sample bottle was homogenised by shaking 100 times by hand. A sample was taken and centrifuged in the normal crude oil laboratory at medium rate for 10 minutes. The free water was pipetted off the bottom of the centrifuge tube. The oil plus some emulsions retained therein (emulsions retained because these contain naphthenic acids) were reshaken in the centrifuge tubes for 25 handshakes.

### (1.3) Comparative Test

### No Inhibitor

Three types of blank tests (uninhibited samples) were performed by methods known in the art, as set out below.
a) the Total Acid Number-TAN (residual neutralizer test) after heating and 15 minutes retention time
b) the Total Acid Number-TAN (residual neutralizer test) after heating and 25 minutes retention time
c) the residual Naphthenate soap forming acids after the 25 minutes heating

The TAN in a) and b) were performed after heating the crude oil samples as prepared in section 1.2 above. A standard automatic electrometric titrator was used with 0.1N alcoholic potassium hydroxide (KOH) solution as the Naphthenic acid neutralizer. A known weight of crude oil was heated on a hotplate at 45 deg C. and under a Nitrogen blanket for the varying times above (sec 1.3, a). The normal solvent as per ASTM D664 method, for this titration was added to the crude oil and titrated by automatic delivery of the KOH neutralizer. The millivolts readout v millitres KOH were recorded, and the amount of neutralizer required is calculated as follows:

mg Neutralizer/gram oil=(titration mls at end point×56.1×0.1)/weight of crude oil (grams)

0.1=Normality of KOH

56.1=Molecular weight of KOH.

Residual Naphthenate Soap Forming Acids after Inhibition

The amount of residual naphthenate soap forming acids remaining after inhibition were measured by methods known to the skilled man (by a Modified IER Method as per ref #10).

### 1.4) Performing the Inhibition Tests

The 3 different crude oil samples as prepared were treated with the inhibitor composition of Example 1.1, above, according to the present technology. The three sets of tests as stated above in Sections 1.1 to 1.3 were repeated. The results are shown in the following Tables:

Table 1 shows the Inhibitor effects on Oil #1 using 50 to 100 ppm of Inhibitor Composition, which resulted in 73 and 90% inhibition at 45 deg C. and 15 minutes.

TABLE 1
I.

| | ppm Inhibitor | mg | | |
|---|---|---|---|---|
| OIL | Composition | Neutralizer/g | % Inhibition | Conditions |
| Oil #1 | 0 | 0.77 | | 45 C./15 mins |
| | 50 | 0.21 | 73 | ″ |
| | 100 | 0.074 | 90 | ″ |

Table 2 illustrates the inhibitor effects on Oil H at the same temperature (45 deg C.), but increasing the time to 25 mins. Only one dosage was used at 125 ppm inhibitor

TABLE 2
II.

| | ppm Inhibitor | mg | | |
|---|---|---|---|---|
| OIL | Composition | Neutralizer/g | % Inhibition | Conditions |
| Oil H | 0 | 3.7 | | 45 C./25 mins |
| | 125 | 1.5 | 59.6 | ″ |

Table 3 graph #3 show for Oil H the effects of the variation of inhibitor as dosage is changed from 50 to 200 pm in steps of 50 ppm. The conditions were 45 deg C. over 15 minutes.

TABLE 3
III.

| | ppm Inhibitor | mg | | |
|---|---|---|---|---|
| OIL | Composition | Neutralizer/g | % Inhibition | A. Conditions |
| Oil H | 0 | 3.7 | | 45 C./15 mins |
| | 50 | 3.2 | 14 | ″ |
| | 100 | 2.48 | 33 | ″ |
| | 150 | 2.3 | 38 | ″ |
| | 200 | 0.97 | 74 | ″ |

Table 4 shows the effects of the inhibitor on the high Mw Naphthenic acids from Oil #I, called the “Deposit forming Soap acids”, only. 25 ppm and 50 ppm Inhibitor resulted in 54% and 78% efficiencies respectively, using the extraction of these acids via the hexane-acetone and Modified IER method.

TABLE 4
IV.

| | | milligrams of | | |
|---|---|---|---|---|
| | ppm Inhibitor | Deposit Soap | | |
| OIL I | Composition | Forming acids | % Inhibition | Conditions |
| Oil I | 0 | 29.2 | | 45 C./15 mins |
| | 25 | 13.5 | 53.8 | ″ |
| | 50 | 6.4 | 78.1 | ″ |

These results demonstrate that the inhibitor inhibits the deposits caused by the naphthenate soap forming acids. Similar inhibition is expected with the compositions of Examples 1.1a, 1.1b and 1.1c.

In a further example of the present technology, a method for the inhibition of deposit forming naphthenic acid/soaps in oil is performed. This includes a step of analyzing the crude oil or water for the acid values (TANs), and % naphthenic acids content, by the known IER method [10]; this ensures the deposit is derived from naphthenic acids. A normal FTIR scan is generally sufficient. If analysis indicates there is a high probability of deposit naphthenate soap forming reactions in the process; or confirms that the system is having, or likely to have, problems with naphthenate soaps, a sample of manifold crude oil is treated with an inhibitor composition (e.g. that set out in Example 1.1 or Example 1.1a above). The naphthenate soap forming acids and mg neutralizer required to combat the residual acids are determined from the sample—e.g. by the methods above. The inhibitor composition may then be applied to the bulk hydrocarbon in the appropriate amount to inhibit/reduce naphthenate soap formation, by methods well known in the art.

1. A composition comprising a at least one component and at least one dispersant, said component having a Total Base Number of 50 mg KOH/g or above, and a substituted primary amine.

2. The composition of claim 1 further comprising a solvent.

3. The composition of claim 1 wherein the substituted primary amine is of general formula: 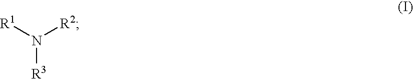
wherein R¹ and R² are H, and R³ is a linear or branched alkyl chain.

4. The composition of claim 3, wherein the linear or branched alkyl chain R³ contains between 1 and 100 Carbon atoms.

5. The composition of claim 3, wherein the linear or branched alkyl chain R³ contains one or more heteroatoms.

6. The composition of claim 3, wherein the linear or branched alkyl chain R³ is optionally substituted.

7. The composition of claim 1, wherein the substituted primary amine is of general formula: 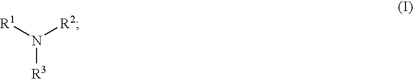
wherein R¹ and R² are H, and R³ is a polyalkylene-polyamine or a polyalkylene-polyamine-hydrocarbyl succinimide residue.

8. The composition of claim 7 the polyalkylene-polyamine-hydrocarbyl succinimide residue is of the general formula:
R⁴—CH₂—CH₂—(NH—CH₂—CH₂)n;
wherein R⁴ is a polyisobutenyl succinimide (PIBS) residue, and n is of the range from 1 to 15.

9. The composition of claim 1, wherein the substituted primary amine is a modified tris succinimide of general formula: 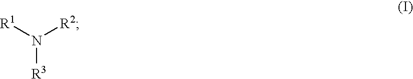
wherein:
R¹ and R² are each of the general formula: R⁴—(NH—CH2-CH2-NH—CH2-CH2-)n;
wherein R⁴ is a polyisobutenyl succinimide residue and n is of the range from 1 to 10; and
R³ is of the general formula: R^{4′}—(NH—CH2-CH2-)n; wherein:
R^{4′} is H and n is of the range from 1 to 10.

10. The composition of claim 3, wherein the substituted primary amine is a modified tris succinimide of general formula: 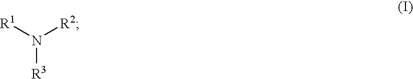
wherein:
R¹ and R² are each of the general formula: R⁴—(NH—CH2-CH2-NH—CH2-CH2-)n;
wherein R⁴ is a polyisobutenyl succinimide residue and n is of the range from 1 to 10; and
R³ is of the general formula: R^{4′}—(NH—CH2-CH2-)n; wherein:
R^{4′} is H and n is of the range from 1 to 10.

11. The composition of claim 7, wherein the substituted primary amine is a modified tris succinimide of general formula: 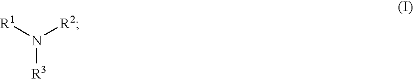
wherein:
R¹ and R² are each of the general formula: R⁴—(NH—CH2-CH2-NH—CH2-CH2-)n;
wherein R⁴ is a polyisobutenyl succinimide residue and n is of the range from 1 to 10; and
R³ is of the general formula: R⁴—(NH—CH2-CH2-)n; wherein:
R^{4′} is H and n is of the range from 1 to 10.

12. The composition of claim 3, wherein the substituted primary amine is a modified tris succinimide of general formula: 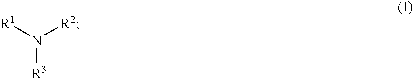
wherein:
R¹ and R² are each of the general formula: R⁴—(NH—CH2-CH2-NH—CH2-CH2-)n; where R⁴ is a polyisobutenyl succinimide residue and n is between 1 and 10; and
R³ is of the general formula R^{4′}—(NH—CH2-CH2-)n; wherein:
R^{4′} is H and n is of the range from 1 to 10.

13. The composition of claim 1 wherein said component further comprises a substituted primary amine having a Total Base Number (TBN) of less than between 50 mg KOH/g; and at least one further booster amine which increases the TBN value to above 50 mg KOH/g.

14. The composition of claim 13 wherein the booster amine is of the general formula: 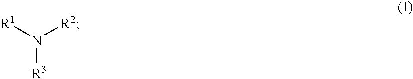
wherein:
R¹ and R² are Hydrogen; and
R³ is of the formula: R⁴—(NH—CH₂—CH₂)n; wherein
R⁴ is H and n is in the range 1-10.

15. The composition of claim 14 wherein n=4.

16. The composition of claim 1, wherein the dispersant is a bis-succinimide of the general formula: 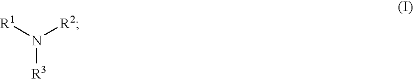
wherein:
R¹ is H; and
R2 and R3 are of formula R⁴—(NH—CH2-CH2-)n; wherein
R⁴ is a polyisobutenyl succinimide residue and n is of the range from 1 to 10.

17. A method of inhibiting or reducing napthenate soap formation in a hydrocarbon composition comprising the step of inhibiting or reducing the reaction of napthenic acids in the hydrocarbon composition by introducing one or more inhibitor compositions comprising a substituted primary amine and having a Total Base Number of at least 50 mg KOH/g.

18. The method of claim 17, wherein the substituted primary amine is of general formula: 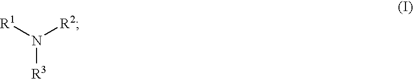
wherein R¹ and R² are H, and R³ is a linear or branched alkyl chain.

19. A method of treating a hydrocarbon composition comprising a step of reacting the hydrocarbon composition with an inhibitor composition comprising a substituted primary amine, said inhibitor composition having a Total Base Number of at least 50 mg KOH/g.

20. The method of claim 19, wherein said method inhibits or reduces naphthenate soap formation due to reaction of naphthenic acids within the hydrocarbon composition.

21. The method of treating a hydrocarbon composition of claim 19, wherein the substituted primary amine is of general formula: 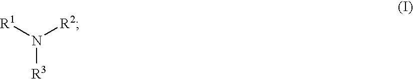
wherein R¹ and R² are H, and R³ is a linear or branched alkyl chain.

22. The method of claim 19, wherein said method reduces the amount of or breaks down the naphthenate soap in the hydrocarbon composition.

23. The method of treating a hydrocarbon composition of claim 19, wherein the substituted primary amine is of general formula: 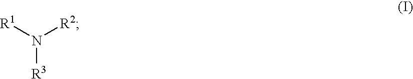
wherein R¹ and R² are H, and R³ is a linear or branched alkyl chain.

24. A method for detecting or quantifying formation of naphthenate soap in a hydrocarbon composition comprising the steps of:
a) applying a stainless steel substrate to the hydrocarbon composition; and
b) measuring the amount of napthenate soap deposited on the stainless steel substrate.
